(19) United States
(12) Patent Application Publication
(10) Pub. No.: US 2023/0390234 A1
(43) Pub. Date: Dec. 7, 2023

(54) COMPOSITION INHIBITING MUSCLE LOSS OR PROMOTING MUSCLE FORMATION THROUGH SKIN-DERIVED EXOSOMES

(71) Applicant: EX HEALTHCARE INC., Gyeonggi-do (KR)

(72) Inventor: Yonghee LEE, Gyeonggi-do (KR)

(21) Appl. No.: 17/909,877

(22) PCT Filed: Nov. 25, 2021
(86) PCT No.: PCT/KR2021/017497

§ 371 Date: Sep. 7, 2022

(30) Foreign Application Priority Data
Feb. 26, 2021 (KR) 10-2021-0026492

(51) Int. Cl. A61K 31/205; A61K 36/82; A61K 31/37; A61K 9/19; A61P 21/04

(52) U.S. Cl. CPC A61K 31/205; A61K 36/82; A61K 31/37; A61K 9/19; A61P 21/04

(57) ABSTRACT

The present invention relates to a composition that increases the level of microRNA-26a in an extracellular vesicle and a use thereof, the composition increases the level of microRNA-26a (miRNA-26a) in an exosome secreted from cells. And the exosome reduces the expression of MURF1, atrogin-1, and myostatin, which are biomarkers involved in muscle loss, and may increase the expression of myoD which is involved in myogenesis. Therefore, the composition, which increases the level of microRNA-26a in the vesicle, may be utilized for a use of suppressing muscle loss and promoting myogenesis.

### TECHNICAL FIELD

The present invention relates to a composition which increases the level of microRNA-26a (miRNA-26a) in an extracellular vesicle and a composition including the above composition and exhibiting an effect of suppressing muscle loss or promoting myogenesis through skin-derived exosomes.

### BACKGROUND ART

Muscles not only act as a human motor ability organ, but also affect the entire body such as bones and blood vessels, nerves, the liver, the heart, and the pancreas. Since bones maintain their density by the force of being pulled and pushed by muscles, bones are also weakened when muscles lose strength, so osteoporosis easily occurs. Further, when muscles are reduced, the effects of various materials produced from muscles interfere with the formation of new blood vessels and nerves, and may ultimately lead to cognitive decline.

Muscle loss or muscle reduction is a lifelong process that begins at about 30 years of age, and muscle tissue mass and the number and size of muscle fibers gradually decrease during this process. The result of muscle loss is a gradual loss of muscle mass and muscle strength. Mild muscle loss increases stress on some joints (for example: the knees) and may cause an individual to be vulnerable to arthritis or falls. In addition, muscle fibers that contract rapidly are more affected by aging than muscle fibers that contract slowly. Therefore, as aging progresses, it is difficult for the muscles to contract rapidly, thereby causing inconvenience in life.

### DISCLOSURE

### Technical Problem

Under the aforementioned situations, the present inventors confirmed that when fibroblasts are treated with a specific material, the level of microRNA-26a (miRNA-26a) is increased in exosomes secreted from cells, and the exosomes reduce the expression of a gene involved in sarcopenia, but increase the expression of a gene involved in myogenesis.

Therefore, an object of the present invention is to provide a composition which increases the level of microRNA-26a (miRNA-26a) in an extracellular vesicle, including a material selected from the group consisting of betaine, a Camellia japonica flower extract, camelliaside A, myricetin, naringenin, nobiletin, kojyl carboxy dipeptide-23, L-carnosine and copper tripeptide, or a combination thereof.

Another object of the present invention is to provide a composition including the above composition as an active ingredient and exhibiting an effect of suppressing muscle loss or promoting myogenesis through skin-derived exosomes.

### Technical Solution

To achieve the objects, an aspect of the present invention provides a composition which increases the level of microRNA-26a (hereinafter, described as a composition for increasing the level of microRNA-26a) in an extracellular vesicle, including a material selected from the group consisting of betaine, a Camellia japonica flower extract, camelliaside A, myricetin, naringenin, nobiletin, kojyl carboxy dipeptide-23, L-carnosine and copper tripeptide, or a combination thereof as an active ingredient.

Betaine is also called trimethylglycine (TMG), is abundant in plants such as sugar cane, beets, and Chinese wolfberry, is known to have excellent skin moisturizing power, and thus is used as a raw material for cosmetics. Myricetin is used as an antioxidant, a skin conditioning agent, and the like, and narinzenin is also a cosmetic raw material used as a skin conditioning agent.

Kojyl carboxy dipeptide-23 is a compound with the following structure which acts as an antioxidant, a metal ion sequestering agent, and a skin protectant:

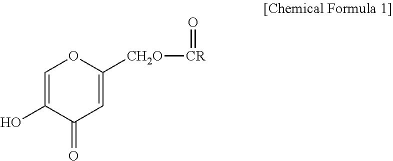

In Chemical Formula 1, R is dipeptide-23.

It is known that copper tripeptide is a copper complex (GHK-Cu) of a tripeptide and used as a skin conditioning agent, and camelliaside A is a component included in green tea (Camellia sinensis) and has an effect of ameliorating wrinkles (Korean Patent No. 10-0757175). Furthermore, it is known that nobiletin is a polymethoxy flavone that is abundant in citrus peel and has an excellent anti-inflammatory effect (Korean Patent No. 2018-0046245).

L-carnosine is a dipeptide including two amino acids, histidine and alanine, is known to have antioxidant and antidiabetic activities, and thus is used as a nutrient and a supplement.

As described above, since the materials used in the composition of the present invention are raw materials for cosmetics and nutritional supplements or derived from natural products, it is safe to use them in the human body.

As used herein, the term “extract” includes an extract solution obtained by extraction treatment, a diluted solution or concentrated solution of the extract solution, a dried product obtained by drying the extract solution, a crude purified product or purified product of the extract solution, or a mixture thereof and the like, an extract solution itself and an extract of all formulations which can be formed using an extract solution. The extract of the present invention may be extracted from natural, hybrids or varieties of each of the corresponding plants, and can also be extracted from a plant tissue culture.

The method for extracting the Camellia japonica flower extract is not particularly limited, and the Camellia japonica flower extract may be extracted by a method typically used in the art. Non-limiting examples of the extraction method include a solvent extraction method, a hot water extraction method, an ultrasonic extraction method, a filtration method, a reflux extraction method and the like, and these may be performed either alone or in a combination of two or more thereof.

An extraction solvent used for the Camellia japonica flower extract is not particularly limited, and any solvent known in the art may be used. Specifically, the Camellia japonica flower extract may be extracted by any one solvent selected from the group consisting of water, ethyl acetate, dichloromethane, an alcohol having 1 to 4 carbon atoms and a combination thereof, and preferably, may be extracted using ethanol as a solvent.

A liquid Camellia japonica flower extract may be separated from a dried shredded plant by a method such as vacuum filtration, and then subjected to a process of concentration or drying. For example, the liquid extract may be a concentrated solution concentrated under reduced pressure with a vacuum rotary concentrator, and a powdered extract may also be obtained by drying the liquid extract. The extract thus concentrated or powdered may be used by being soluble in water, alcohol, dimethyl sulfoxide (DMSO) or a mixed solvent thereof, as necessary.

The present inventors confirmed that when skin fibroblasts were treated with the material, and then exosomes were isolated, the level of microRNA-26a (miRNA-26a) in the exosomes was increased compared to the level when skin fibroblasts were not treated with the material (Table 1). Therefore, the extracellular vesicle is not limited thereto, but may be secreted from fibroblasts, specifically skin fibroblasts.

As used herein, the term “extracellular vesicle” refers to an extracellular-released vesicle with a very small size, which serves as a mediator of physiological signaling by enabling materials such as proteins, lipids and nucleic acids to be exchanged between cells, and almost all cells secrete extracellular vesicles. Extracellular vesicles are broadly classified into exosomes and microvesicles according to their size and production process. Referring to the production process, in exosomes, vesicles are produced inside cells, and thus secreted while the cell membrane is folded inward, and have a size of approximately 30 to 200 nm, and microvesicles are secreted outside the cells while the cell membrane protrudes outward and is separated, and have a size of approximately 50 to 1,000 nm.

According to an exemplary embodiment of the present invention, the extracellular vesicle may be an exosome.

According to exemplary embodiments of the present invention, the composition for increasing the level of microRNA-26a may include an active ingredient (a material that promotes the secretion of skin-derived exosomes) in an amount of 0.00001 wt % or more with respect to the total weight of the composition. More specifically, the active ingredient may be included in an amount of 0.00001 wt % or more, 0.0001 wt % or more, 0.0005 wt % or more, 0.001 wt % or more, 0.005 wt % or more, 0.01 wt % or more, 0.05 wt % or more, 0.1 wt % or more, 0.5 wt % or more, 1.0 wt % or more, 5.0 wt % or more, 10 wt % or more or 50 wt % or more, and 70 wt % or less, with respect to the total weight of the composition. Preferably, the composition may include the active ingredient in an amount of 0.00001 wt % to 10 wt % or 0.005 to 10 wt %.

Another aspect of the present invention provides a composition for suppressing muscle loss or promoting myogenesis, including the composition for increasing the level of microRNA-26a as an active ingredient.

As already described, when cells, specifically, skin cells are treated with the composition for increasing the level of microRNA-26a, extracellular vesicles with an increased level of microRNA-26a, specifically, exosomes, may be obtained.

Meanwhile, it is well known that exosomes are used as a means of intercellular communication. For example, exosomes secreted from stem cells in bones arrive at the heart to transmit signals. Compared to the past when it was known that hormones are mainly responsible for signaling between organs in the body, it has been recently revealed that exosomes are also used for signaling between organs. Since the skin is the largest organ in the body, exosome secretion is expected to be very active, but very little research has been done on the role of exosomes secreted from the skin.

Such a communication function of exosomes may be confirmed even in the present invention. Specifically, although direct treatment of myofibroblasts with a material such as betaine did not significantly change the expression of muscle loss- and myogenesis-related genes, it was confirmed that when exosomes with an increased level of miRNA-26a obtained by the treatment with the material were treated, the expression of muscle loss-related genes was reduced and the expression of myogenesis-related genes was increased to significant levels (FIGS. 4 to 7, 9 and 10). These experimental results mean that exosomes derived from fibroblasts and with an increased level of miRNA-26a are involved in myogenesis promotion and muscle loss suppression-related signaling in myofibroblasts.

The muscle loss-related gene is selected from the group consisting of muscle ring-finger protein 1 (MURF1), atrogin-1 and myostatin, and the myogenesis-related gene may be myogenic differentiation 1 (myoD).

The composition for suppressing muscle loss or promoting myogenesis of the present invention may be prepared in any formulation typically prepared in the art, and may be formulated as, for example, a solution, a suspension, an emulsion, a paste, a gel, a cream, a lotion, a powder, a powdered foundation, an emulsion foundation, a wax foundation, a spray, or the like, but not limited thereto. More specifically, the composition of the present invention acts on fibroblasts, and thus may have a cream, lotion, ointment or gel formulation, and may be used in the form of an external skin preparation. Compositions in such formulations may be prepared by a typical method in the related art.

The composition for suppressing muscle loss or promoting myogenesis of the present invention may additionally include an ingredient included in a general cosmetic composition in addition to the active ingredient. Examples of blended ingredients which may be added include a moisturizer, an emollient, a surfactant, organic and inorganic pigments, an organic powder, an ultraviolet absorbent, a preservative, a bactericide, an antioxidant, a plant extract, a pH adjuster, an alcohol, a colorant, a fragrance, a circulation accelerator, a cooling agent, an antiperspirant, purified water, and the like.

When the formulation of the present invention is a cream or a gel, an animal fiber, a vegetable fiber, a wax, paraffin, starch, traganth, a cellulose derivative, polyethylene glycol, silicone, bentonite, silica, talc, zinc oxide, or the like may be used as a carrier ingredient.

When the formulation of the present invention is a powder or a spray, lactose, talc, silica, aluminum hydroxide, calcium silicate, or a polyamide powder may be used as the carrier ingredient, and in particular, when the formulation of the present invention is a spray, the formulation may additionally include a propellant such as a chlorofluorohydrocarbon, propane/butane or dimethyl ether.

When the formulation of the present invention is a solution or an emulsion, a solvent, a solubilizer or an emulsifier is used as the carrier ingredient, and examples thereof include water, ethanol, isopropanol, ethyl carbonate, ethyl acetate, benzyl alcohol, benzyl benzoate, propylene glycol, 1,3-butylglycol oil, glycerol aliphatic esters, polyethylene glycol or fatty acid esters of sorbitan.

When the formulation of the present invention is a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, traganth, or the like may be used as the carrier ingredient.

Still another aspect of the present invention provides a pharmaceutical composition for preventing or treating a muscle reduction-related muscle disease, including the composition for increasing the level of microRNA-26a as an active ingredient.

The muscle reduction-related muscle disease may be selected from the group consisting of sarcopenia, muscular atrophy, muscular dystrophy and myasthenia.

Sarcopenia refers to a disease in which normal muscle mass, muscle strength and muscle function are reduced due to malnutrition, decreased exercise, aging, and the like, and muscular atrophy refers to a group of clinically and genetically diverse diseases in which symmetrical muscle weakness or loss appears for reasons such as heredity.

Muscular dystrophy (MD) or myodystrophia is a muscular disease that weakens locomotion and interferes with motor performance, and is characterized by progressive weakening of skeletal muscle, deficiency of muscle proteins, and necrosis of muscle cells and tissues. Myasthenia is a disease in which muscle strength is abnormally weakened or fatigued, and when myasthenia is not properly treated, muscle weakness may suddenly become severe, and in severe cases, respiratory muscles may also be weakened, leading to respiratory paralysis.

Among the terms or elements referred to in the pharmaceutical composition, those referred to in the description on the composition for increasing the level of microRNA-26a are understood as those mentioned in the description on the claimed composition for increasing the level of microRNA-26a.

The pharmaceutical composition of the present invention may comprise a pharmaceutically acceptable carrier in addition to the active ingredient. In this case, the pharmaceutically acceptable carrier is typically used during formulation, and includes lactose, dextrose, sucrose, sorbitol, mannitol, starch, acacia rubber, calcium phosphate, alginate, gelatin, calcium silicate, microcrystalline cellulose, polyvinylpyrrolidinone, cellulose, water, syrup, methylcellulose, methyl hydroxybenzoate, propyl hydroxybenzoate, talc, magnesium stearate, mineral oil, and the like, and is not limited thereto. Furthermore, pharmaceutical composition of the present invention may further include a lubricant, a wetting agent, a sweetening agent, a flavoring agent, an emulsifier, a suspending agent, a preservative, and the like in addition to the above ingredients.

The pharmaceutical composition of the present invention may be administered orally or parenterally (for example, skin application, intravenous, subcutaneous, intraperitoneal injection or applied topically) according to the target method. Preferably the pharmaceutical composition of the present invention may be administered parenterally.

When the active ingredient of the present invention is formulated into a preparation such as tablets, capsules, chewable tablets, a powder, a liquid, and a suspending agent for the purpose of oral administration, it is possible to comprise a binder such as arabic rubber, corn starch, microcrystalline cellulose or gelatin, an excipient such as calcium diphosphate or lactose, a disintegrant such as alginic acid, corn starch, or potato starch, a lubricant such as magnesium stearate, a sweetening agent such as sucrose or saccharin, and a flavoring agent such as peppermint, methyl salicylate, or fruit flavor.

Furthermore, the parenteral administration form may be a transdermal administration type-formulation, and may be a formulation such as, for example, an injection, an adhesive, an ointment, a lotion, a gel, a cream, a spray, a suspending agent, an emulsion, a suppository, or a patch, but is not limited thereto.

Further, the pharmaceutical composition may be in the form of an external skin preparation, and the external skin preparation is a general term that can include anything applied from the outside of the skin, and any pharmaceutical product having various dosage forms may be included herein.

The pharmaceutical composition of the present invention is administered in a pharmaceutically effective amount. In the present invention, the “pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and other factors well known in the medical field. For example, the pharmaceutical composition of the present invention may be administered in a dose of 1 μg/kg to 200 mg/kg, preferably 50 μg/kg to 50 mg/kg once a day or three times a day in divided doses. The dose is not intended to limit the scope of the present invention in any way.

The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by a person skilled in the art.

Yet another aspect of the present invention provides a composition (hereinafter, described as an extracellular vesicle composition) including, as an active ingredient, extracellular vesicles obtained by treating cells with a material selected from the group consisting of betaine, a Camellia japonica flower extract, camelliaside A, myricetin, naringenin, nobiletin, kojyl carboxy dipeptide-23, L-carnosine and copper tripeptide, or a combination thereof, and a composition for suppressing muscle loss or promoting myogenesis, including the extracellular vesicle composition as an active ingredient.

The present inventors confirmed that when skin-derived fibroblasts were treated with the above-described materials, the level of microRNA-26a was increased in exosomes secreted from fibroblasts, and confirmed that the exosomes had an effect of suppressing muscle loss or promoting myogenesis (FIGS. 4 to 7, 9 and 10).

Among the terms or elements referred to in the extracellular vesicle composition, those referred to in the description on the composition for increasing the level of microRNA-26a are understood as those mentioned in the description on the claimed composition for increasing the level of microRNA-26a.

Yet another aspect of the present invention provides a method for producing extracellular vesicles with an increased level of microRNA-26a, including the following steps:
treating cells with a material selected from the group consisting of betaine, a Camellia japonica flower extract, camelliaside A, myricetin, naringenin, nobiletin, kojyl carboxy dipeptide-23, L-carnosine and copper tripeptide, or a combination thereof; and
recovering extracellular vesicles from a cell culture medium.

According to an exemplary embodiment of the present invention, the cells may be skin-derived fibroblasts, and the extracellular vesicles may be exosomes, but are not limited thereto.

Meanwhile, the method for isolating extracellular vesicles and/or exosomes from the cell culture medium is as disclosed in Example 1-2. However, as the method for isolating extracellular vesicles and/or exosomes from a cell culture medium, various methods known in the art may be used in addition to the isolation method described above.

For example, in order to isolate extracellular vesicles and/or exosomes, it is possible to use a known isolation method such as ultrafiltration, density gradient centrifugation, tangential flow filtration, size exclusion chromatography, ion exchange chromatography, immunoaffinity capture, microfluidics-based isolation, exosome precipitation, or polymer-based precipitation. However, the exosome isolation method is not limited to the methods described above, and it goes without saying that various separation methods which are used in the art and may be used in the future can be adopted.

### Advantageous Effects

When a composition which increases the level of microRNA-26a in the extracellular vesicle according to an exemplary embodiment of the present invention is used, it is possible to prepare exosomes with an increased level of microRNA-26a. Since the exosomes can reduce the expression of MURF1, atrogin-1, and myostatin, which are biomarkers involved in muscle loss, and can increase the expression of myoD involved in myogenesis, the composition which increases the level of microRNA-26a in extracellular vesicles can be usefully used for a use of suppressing muscle reduction and muscle loss or promoting myogenesis.

### DESCRIPTION OF DRAWINGS

FIG. 1A illustrates the result of confirming cell viability after fibroblasts are treated with betaine at different concentrations.

FIG. 1B illustrates the result of confirming cell viability after myofibroblasts are treated with betaine at different concentrations.

FIG. 2 illustrates the results of treating myofibroblasts with fibroblast-derived exosomes labeled with fluorescence and then confirming whether they are absorbed into the cells.

FIG. 3 illustrates the results of confirming the level of miRNA-26a in fibroblast-derived exosomes isolated by treatment with betaine at different concentrations.

FIG. 4A illustrates the result of treating myofibroblasts with betaine or fibroblast-derived exosomes, and then confirming the expression level of muscle loss marker MURF1.

FIG. 4B illustrates the result of treating myofibroblasts with betaine or fibroblast-derived exosomes, and then confirming the expression level of muscle loss marker atrogin-1.

FIG. 4C illustrates the results of treating myofibroblasts with betaine or fibroblast-derived exosomes, and then confirming the expression level of muscle loss marker myostatin.

FIG. 5 illustrates the results of treating myofibroblasts with betaine or fibroblast-derived exosomes, and then confirming the expression level of a myogenesis marker MyoD.

FIG. 6 illustrates the results of treating myofibroblasts with betaine or fibroblast-derived exosomes, and then confirming the protein levels of muscle loss markers MURF1, atrogin-1, and myostatin.

FIG. 7 illustrates the results of treating myofibroblasts with betaine or fibroblast-derived exosomes, and then confirming the protein level of a myogenesis marker MyoD.

FIG. 8 illustrates the results of treating myofibroblasts with dexamethasone, and then confirming the expression levels of MURF1 and MyoD according to the treatment concentration of a miRNA-26a mimic.

FIG. 9 illustrates the results of treating myofibroblasts with a Camellia japonica flower extract, and then confirming the level of miRNA-26a in the isolated exosomes.

FIG. 10A illustrates the result of treating myofibroblasts with a Camellia japonica flower extract or exosomes isolated from fibroblasts treated with the Camellia japonica flower extract, and then confirming the expression level muscle loss marker MURF1.

FIG. 10B illustrates the result of treating myofibroblasts with a Camellia japonica flower extract or exosomes isolated from fibroblasts treated with the Camellia japonica flower extract, and then confirming the expression level of muscle loss marker myostatin.

### MODES OF THE INVENTION

Hereinafter, one or more specific exemplary embodiments will be described in more detail through Examples. However, these Examples are provided only for exemplarily explaining the one or more specific exemplary embodiments, and the scope of the present invention is not limited to these Examples.

### Example 1: Isolation and Analysis of Exosomes

1-1. Confirmation of Cytotoxicity of Betaine

As human skin dermal cells, normal human dermal fibroblasts (NHDF, fibroblasts) isolated from adult samples were purchased from LONZA (Cat. CC-2511). As myofibroblasts, C2C12 cells, which are myoblasts isolated and cultured from C3H mice, were purchased from ATCC (CRL-1772).

Subcultured passage 27 human fibroblast HS68 (hereinafter referred to as FB) or C2C12 was inoculated onto a 96-well plate at a concentration of 1×10⁴ cells/well and cultured in an incubator at 5% CO₂ and 37° C. for 24 hours. After culturing, cells were treated with betaine at different concentrations and additionally cultured for 24 hours, and an untreated group was used as a control. After the cells were washed with PBS, the cells were additionally cultured with CCK-8 (DONGJIN, CK04-11) for 2 hours, and absorbance was measured at 450 nm using a microplate reader. From the measured results, relative cell viability according to the betaine treatment concentration was calculated by setting the value of the control to 100.

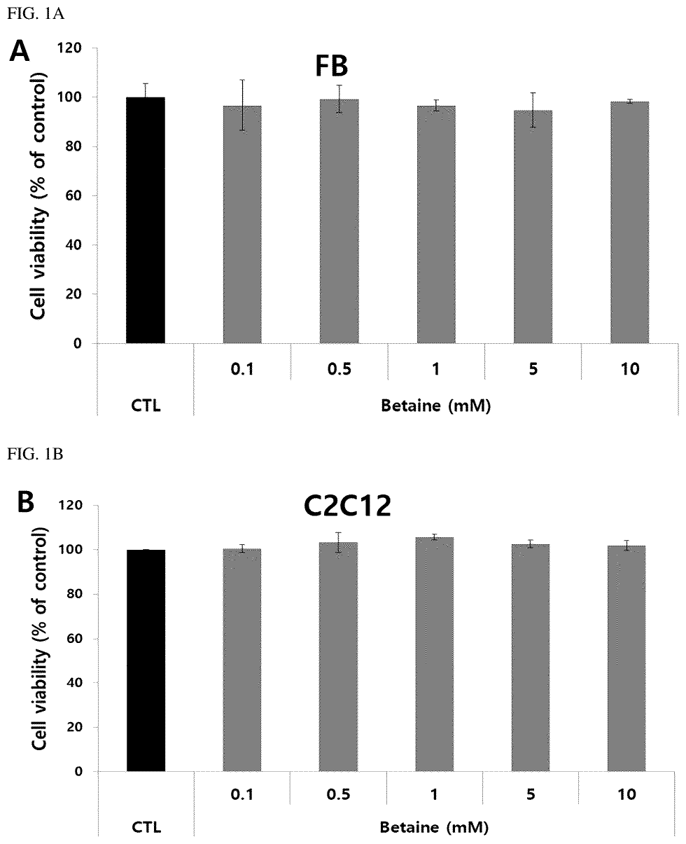

As a result, it was confirmed that there was no change in the viability of FB and C2C12 even after treatment with betaine (FIGS. 1A and 1B).

1-2. Isolation of Exosomes

After treating the subcultured passage 27 FB with betaine for 48 hours, the medium was collected and centrifuged at 3000×g for 30 minutes. After centrifugation, only the supernatant was recovered and transferred to Ultra-15 Centrifugal Filter Units (Amicon, MERCK, C7715) and centrifuged at 4000×g for 40 minutes. Only the supernatant was recovered, Total Exosome Isolation Reagent (Invitrogen, Cat No #4478539) was added at ½ the volume of the supernatant, and the mixture was allowed to react at 4° C. overnight. The next day, after centrifugation at 10000×g for 1 hour, the supernatant was removed by suction, and then a final exosome pellet was re-suspended in PBS. The exosomes obtained in the present example are hereinafter referred to as “fibroblast-derived exosomes”.

In order to confirm the size of the fibroblasts (FB)-derived exosomes, dynamic light scattering was measured using Zetasizer Nano ZS (Malvern Instruments, Worcestershire, UK), and the measurement results were analyzed using Dynamic V6 software. As a result of the analysis, it was found that the size of fibroblast-derived exosomes was in a range of 50 to 150 nm.

Further, absorbance was measured at 405 nm using an EXOCET Exosome Quantitation Kit (System Biosciences, USA) for the quantification of fibroblast (FB)-derived exosomes.

Based on the analysis results, all experimental groups were treated with fibroblast-derived exosomes at a concentration of 20 μg/ml.

1-3. Confirmation of Whether Fibroblast-Derived Exosomes are Absorbed into Cells

It was confirmed whether fibroblast-derived exosomes were absorbed into C2C12 cells. 1.5×10⁴ C2C12 cells were cultured by inoculating 1.5×10⁴ cells onto Lab-Tek chamber slides (Nunc Penfield, NY) and cultured, fibroblast-derived exosomes were treated with a body TR ceramide staining reagent for 20 minutes, and then the excess staining reagent was removed using a cleanup kit. C2C12 cells were treated with fibroblast-derived exosomes labeled with the staining reagent for 30 minutes and then observed under a confocal microscope.

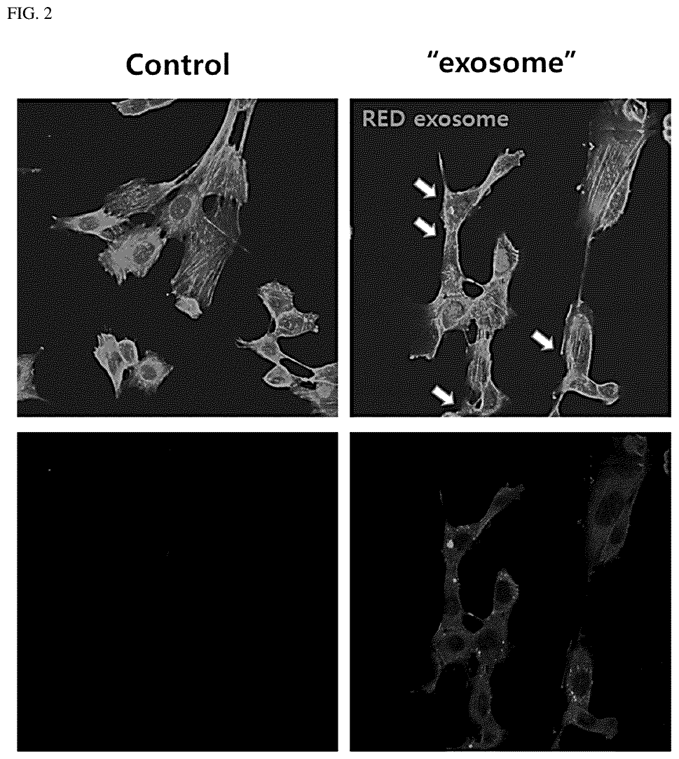

As a result of the observation, it could be seen that fibroblast-derived exosomes were absorbed into the cells because red dot-like signals indicating exosomes could be confirmed in C2C12 cells (FIG. 2).

1-4. Confirmation of Level of miRNA-26a in Fibroblast-Derived Exosomes Treated with Betaine

2×10⁶ FB cells were inoculated onto a 75 T flask, cultured for 24 hours, and then treated with betaine (0.1, 1 and 10 mM). After the cells were additionally cultured for 48 hours, exosomes were isolated by the method of Example 1-2, and microRNA (hereinafter described as miRNA) was extracted from the exosomes isolated from the culture medium using an RNeasy plus mini kit (Qiagen, Germany) according to the manufacturer's protocol.

Real-time qPCR was performed with a tagman probe targeting mature RNA. The relative expression level of the intracellular target miRNA was normalized with the expression level of RNU48, which is used as a housekeeping gene during miRNA quantification, and then shown as a relative %. The relative expression of miRNA-26a in exosomes was normalized with the expression of microRNA-26a, which is used as a housekeeping gene during quantification of miRNA in fibroblast exosomes, and then shown as a relative %. For all real-time qPCR analyses, an Applied Biosystems 7500 (Applied Biosystems) apparatus was used.

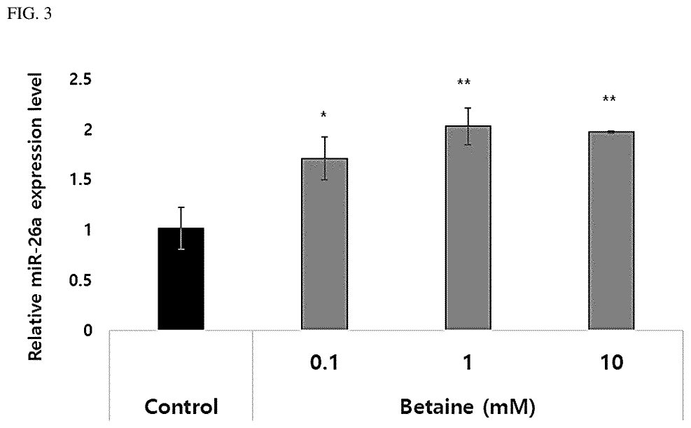

As a result of the analysis, it could be confirmed that the level of miRNA-26a was increased in exosomes isolated from betaine-treated FB cells compared to the control not treated with betaine (FIG. 3).

1-5. Additional Isolation of Exosomes

The FB was treated with the material shown in the following Table 1 to isolate exosomes in the same manner as in Example 1-1, and the level of miRNA-26a was confirmed in the exosomes by the method in Example 1-4.

TABLE 1

| | | | | fold change | |
|---|---|---|---|---|---|
| Classification | Material name | Concentration | | (vs. control) | p-value |
| peptide | Kojyl carboxy dipeptide-23 | 10 | ppm | 1.483 | * |
| | (Creative BioMart, COS-515) | | | | |
| | Copper Tripeptide | | | 1.743 | ** |
| | (Biosynth carbosynth, FC138108) | | | | |
| | L-carnosine | 5 ppm | | 1.537 | * |
| | (Sigma Aldrich, C9625) | (0.0005%) | | | |
| Chemical | Betaine | 1 | mM | 1.821 | ** |
| | (Sigma Aldrich, W422312) | | | | |
| | Camelliaside A | 1 | ppm | 1.956 | ** |
| | (Biosynth carbosynth, OC33270) | | | | |
| flavonols | Myricetin | 25 | uM | 1.599 | * |
| | (Sigma Aldrich, M6760) | | | | |
| flavanones | Naringenin (Sigma Aldrich, N5893) | 10 | uM | 1.854 | *** |
| Flavones | Nobiletin (Sigma Aldrich, N1538) | 30 | uM | 1.447 | ** |
| Extract | Camellia japonica flower extract | 50 | ppm | 3.618 | *** |

### Example 2: Confirmation of Changes in Gene Expression of Myofibroblasts

2-1. Confirmation of Changes in Muscle Loss/Myogenesis Marker Expression_qPCR

C2C12 cells were inoculated onto a 6-well plate at a concentration of 1.5×10⁵ cells/well and cultured for 24 hours. Thereafter, the medium was exchanged with a medium supplemented with 2% horse serum, and the cells were further cultured for 72 hours to promote the differentiation of C2C12 cells. After the differentiation was completed, the cells were treated with betaine at 0.1 mM or fibroblast-derived exosomes (about 20 μg/ml) obtained in Example 1-2 and further cultured for 48 hours. Total RNA was extracted with an RNeasy plus mini kit and cDNA was synthesized with SuperScript™ III (Invitrogen, USA). Thereafter, qPCR was performed with a target gene taqman probe, and then analysis was performed.

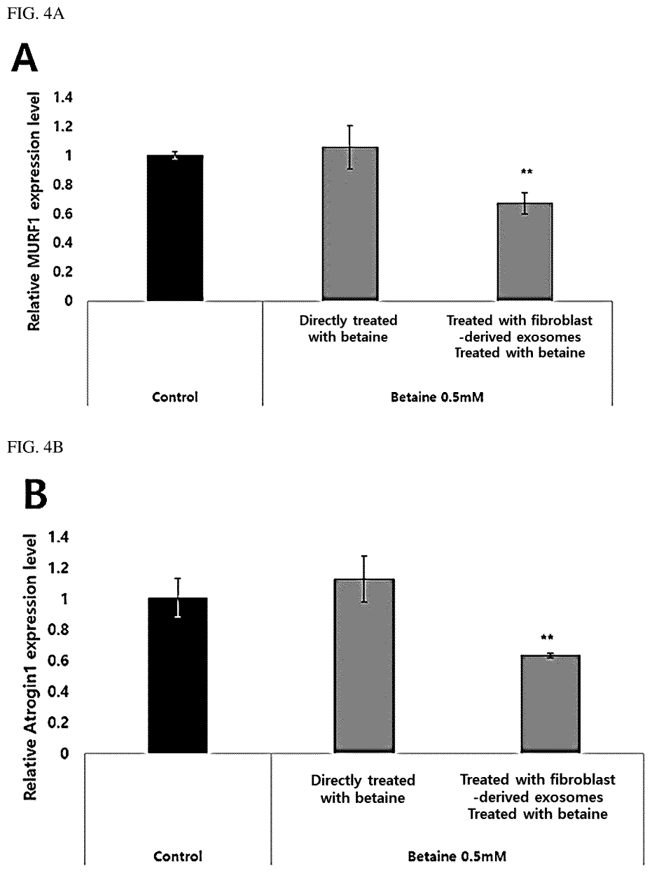

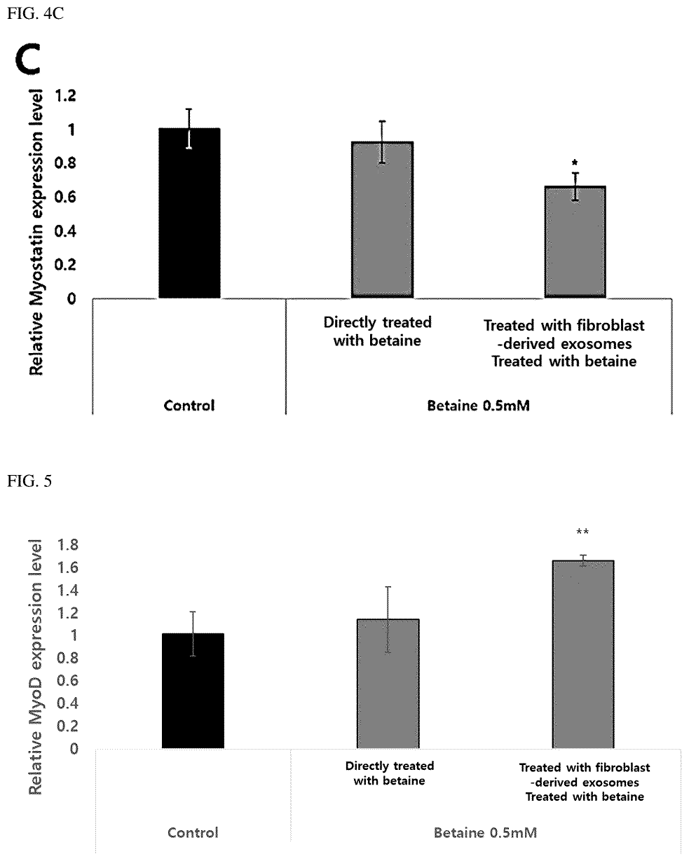

As a result of the analysis, it was confirmed that when C2C12 cells, which are myofibroblasts, were directly treated with betaine, the expression of muscle ring finger 1 (MURF1) and atrogin-1, which are muscle loss markers, was slightly increased compared to the control, and the expression of myostatin was at almost the same level. However, none of the three genes had a significant level of change. In contrast, it could be seen that treatment of C2C12 cells with fibroblast-derived exosomes reduced the expression of the muscle loss markers MURF1, atrogin-1, and myostatin to significant levels (FIGS. 4A to 4C).

In addition, when C2C12 cells, which are myofibroblasts, were directly treated with betaine, the expression of myogenic differentiation 1 (MyoD), which is a myogenesis marker, was increased compared to the control, but was not at a significant level. In contrast, it could be confirmed that treatment with fibroblast-derived exosomes increased the expression of the myogenesis marker MyoD to a significant level (FIG. 5).

2-2. Confirmation of Changes in Muscle Loss/Myogenesis Marker Expression_Western Blotting

C2C12 cells were inoculated onto a 6-well plate at a concentration of 5×10⁵ cells/well and cultured for 24 hours. Thereafter, the medium was exchanged with a medium supplemented with 2% horse serum, and the cells were further cultured for 72 hours to promote the differentiation of C2C12 cells. After the differentiation was completed, the cells were treated with betaine (0.1 and 0.5 mM) or fibroblast-derived exosomes and further cultured for 48 hours. Thereafter, cells were lysed by adding a lysis buffer (1% NP40, 0.05 M Tris-HCl, pH 7.5, 0.15 M NaCl and 0.01 MgCl₂) including a protease inhibitor to the C2C12 cells, and the concentration of proteins was measured by a bovine carbonic anhydrase (BCA) method. The quantified protein was separated with SDS-PAGE and transferred to a PVDF membrane, and the protein expression level of each muscle loss marker was confirmed with an antibody.

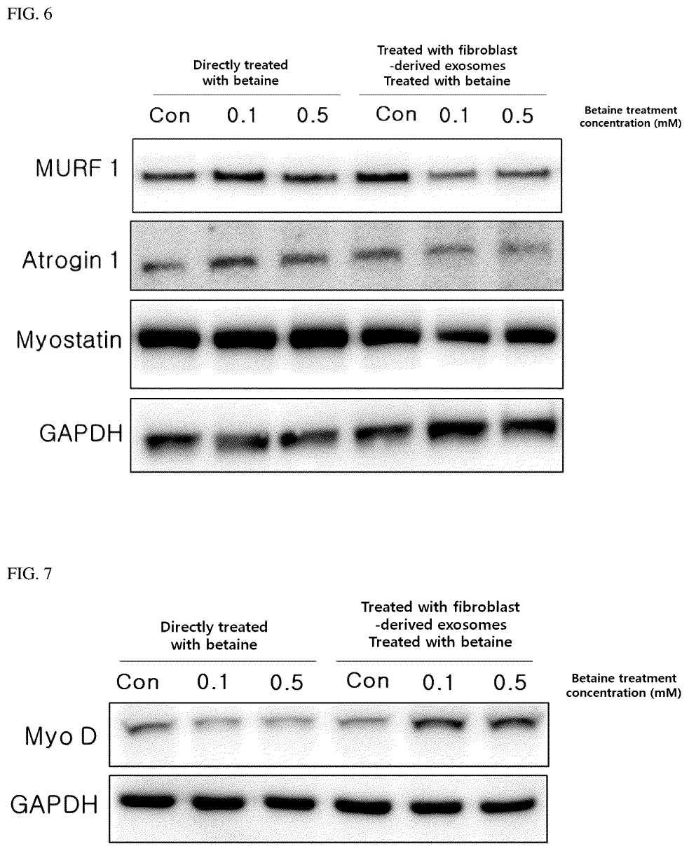

As a result of the confirmation, it could be confirmed that when C2C12 cells were directly treated with betaine at a concentration of 0.1 or 0.5 mM, there was no change in expression of the muscle loss marker, but when C2C12 cells were treated with fibroblast-derived exosomes, the expression of muscle ring-finger protein-1 (MURF1), atrogin-1 and myostatin, which are muscle loss markers, was reduced (FIG. 6). Further, it could be seen that the expression of MyoD, a myogenesis marker, was unchanged when C2C12 cells were directly treated with betaine, but was increased by treatment with fibroblast-derived exosomes (FIG. 7).

### Example 3: Confirmation of Changes in Gene Expression by miRNA-26a

Since it was confirmed that the level of miRNA-26a was increased in fibroblast-derived exosomes by treatment with betaine, the efficacy of miRNA-26a on suppressing muscle loss and promoting myogenesis was confirmed as follows. C2C12 cells were treated with 100 uM of dexamethasone, which is known to induce muscle loss, and cultured by being treated with a miRNA-26a mimic at 10 and 20 nM. Thereafter, changes in the expression of the muscle loss and myogenesis markers were confirmed by western blotting.

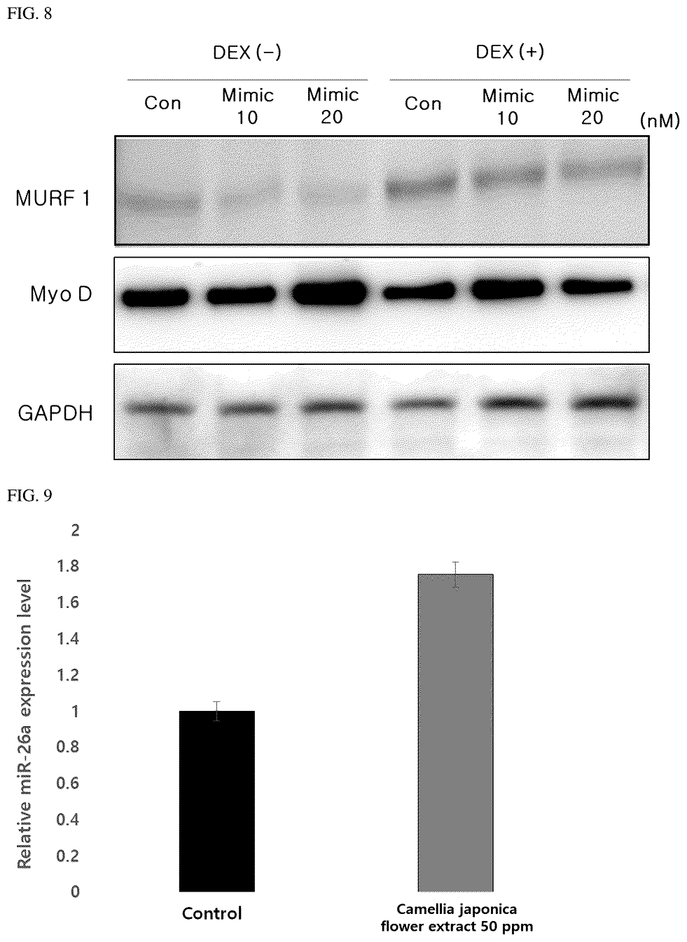

As a result of the confirmation, it could be seen that the expression of MURF1, which is a muscle loss inhibitory marker, was decreased and the expression of MyoD, which is a myogenesis promoting marker, was increased by treatment with the miRNA-26a mimic (FIG. 8).

### Example 4. Confirmation of Efficacy of Exosomes Treated with Camellia japonica Flower Extract

4-1. Preparation of Camellia japonica Flower Extract and Isolation of Exosomes

Camellia japonica flowers were dried at 50° C. overnight using a hot air dryer and crushed. Dried Camellia japonica flowers (100 g) were extracted with 70% (v/v) ethanol at room temperature overnight. After the resulting extract was subjected to a filtration process, the solvent was removed by a rotary vacuum evaporator and the extract was freeze-dried to prepare a Camellia japonica flower extract.

Thereafter, after fibroblasts (FB) were treated with the Camellia japonica flower extract for 48 hours, exosomes were isolated by the method in Example 1-2. Furthermore, the level of miR-26a was confirmed in exosomes isolated from the culture medium by the method in Example 1-4. As a result of the confirmation, it could be confirmed that the level of miRNA-26a was increased in the exosomes isolated from the FB treated with the Camellia flower extract at a concentration of 50 ppm compared to the control which was an untreated group (FIG. 9).

4-2. Confirmation of Changes in Muscle Loss/Myogenesis Marker Expression_qPCR

C2C12 cells were inoculated onto a 6-well plate at a concentration of 1.5×10⁵ cells/well and cultured for 24 hours. Thereafter, the medium was exchanged with a medium supplemented with 2% horse serum, and the cells were further cultured for 72 hours to promote the differentiation of C2C12 cells. After the differentiation was completed, the cells were treated with the Camellia japonica flower extract at 50 ppm or the exosomes (about 20 μg/ml) obtained in Example 4-1 and further cultured for 48 hours. Thereafter, the change in expression of the target gene was confirmed by qPCR by the method in Example 2-1.

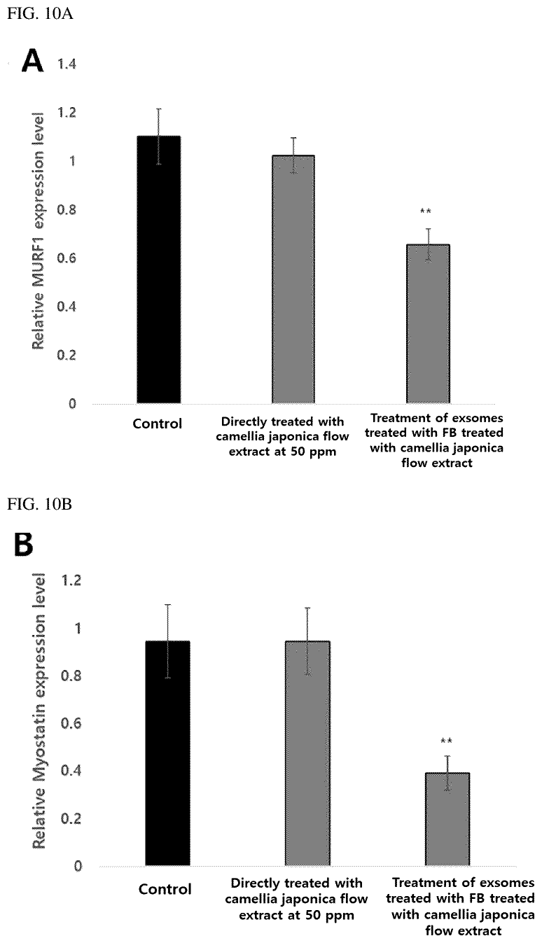

As a result, it could be seen that in an experimental group treated with the exosomes obtained in Example 4-1 compared to the control which was the untreated group and an experimental group directly treated with the Camellia japonica flower extract, the expression of MURF1 and myostatin, which are muscle loss markers, was significantly reduced (FIGS. 10A and 10B).

1. A composition which increases the level of microRNA-26a (miRNA-26a) in an extracellular vesicle, comprising a material selected from the group consisting of betaine, a Camellia japonica flower extract, camelliaside A, myricetin, naringenin, nobiletin, kojyl carboxy dipeptide-23, L-carnosine and copper tripeptide, or a combination thereof.

2. The composition of claim 1, wherein the extracellular vesicle is an exosome.

3. The composition of claim 1, wherein the extracellular vesicle is secreted from fibroblasts.

4. A composition for suppressing muscle loss or promoting myogenesis, comprising the composition of claim 1 as an active ingredient.

5. The composition of claim 4, wherein the composition reduces the expression of muscle loss-related genes selected from the group consisting of muscle ring-finger protein-1 (MURF1), atrogin-1 and myostatin.

6. The composition of claim 4, wherein the composition increases the expression of a myoD gene that promotes myogenesis.

7. The composition of claim 4, wherein the composition is a cream, lotion, ointment or gel formulation.

8. A pharmaceutical composition for preventing or treating a muscle reduction-related muscle disease, comprising the composition of claim 1 as an active ingredient.

9. The composition of claim 7, wherein the muscle reduction-related muscle disease is selected from the group consisting of sarcopenia, muscular atrophy, muscular dystrophy and myasthenia.

10. A composition comprising, as an active ingredient, an extracellular vesicle obtained by treating cells with a material selected from the group consisting of betaine, a Camellia japonica flower extract, camelliaside A, myricetin, naringenin, nobiletin, kojyl carboxy dipeptide-23, L-carnosine and copper tripeptide, or a combination thereof.

11. The composition of claim 10, wherein the cells are fibroblasts, and the extracellular vesicle is an exosome.

12. The composition of claim 10, wherein in the extracellular vesicle, the level of microRNA-26a is increased.

13. A composition for suppressing muscle loss or promoting myogenesis, comprising the composition of claim 1 as an active ingredient.

14. A method for producing an extracellular vesicle in which the level of microRNA-26a is increased, the method comprising: treating cells with a material selected from the group consisting of betaine, a Camellia japonica flower extract, camelliaside A, myricetin, naringenin, nobiletin, kojyl carboxy dipeptide-23, L-carnosine and copper tripeptide, or a combination thereof; and
recovering extracellular vesicles from a cell culture solution.

15. The method of claim 14, wherein the cells are fibroblasts, and the extracellular vesicle is an exosome.
